(19) United States
(12) Patent Application Publication
(10) Pub. No.: US 2007/0297995 A1
(43) Pub. Date: Dec. 27, 2007

(54) Organo-Mineral Sunscreen Composition Adapted for Being Applied by a Propulsion Pump

(75) Inventors: Jean-Christophe Choulot, Rambouillet (FR); Jocelyne Phelouzat, Pierres (FR); Ludovic Guinet, Courbevoie (FR)

Correspondence Address: FOLEY AND LARDNER LLP;SUITE 500, 3000 K STREET NW, WASHINGTON, DC, 20007, US

(73) Assignee: LABORATOIRES EXPANSCIENCE

(21) Appl. No.: 10/585,664

(22) PCT Filed: Jan. 10, 2005
(86) PCT No.: PCT/FR05/00048

§ 371 Date: May 22, 2007

(30) Foreign Application Priority Data
Jan. 9, 2004 (FR) 0400174

(51) Int. Cl. A61K 8/06; A61K 8/25; A61K 8/27; A61K 8/29; B05B 11/00

(52) U.S. Cl. 424/59

(57) ABSTRACT

The invention concerns a cosmetic or dermatological composition for protecting against ultraviolet rays based on a mixture consisting of a mineral screening agent and an organic screening agent having the generic chemical name (INN): methylene bis-benzotriazolyl tetramethylbutylphenol. The invention is characterized in that it exists in the form of a water-in-oil emulsion and that it contains at least one emulsifier selected from the group consisting of siliconized derivatives having a glucosidic constituent containing a number of glucose units between 2 and 10, the particulate inorganic screening agent being dispersed in the water-in-oil emulsion in a homogeneous manner, its average particle size range from 1 to 100 nanometers, and the particulate inorganic screening agent being present in an amount ranging from 4 to 40% by weight.

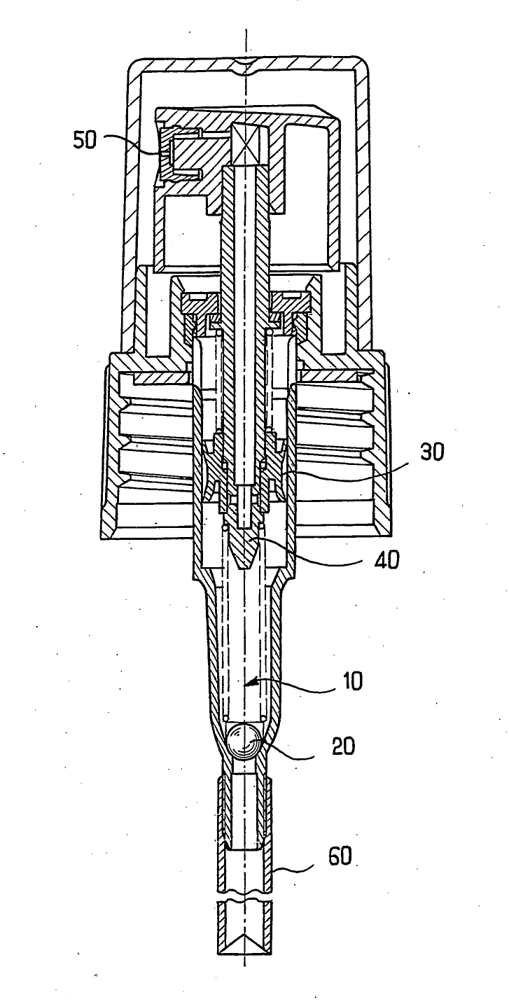

The present invention relates to a cosmetic or dermatological composition based on a mixture of a mineral screening agent and an organic screening agent having the international nonproprietary name (INN) methylenebis(benzotriazolyl)tetramethylbutylphenol, which simultaneously has very good fluidity and excellent broad-spectrum photoprotection. This cosmetic composition is particularly suitable for application to the skin via a propulsion pump.

The invention also relates to a pump device specially designed for the composition according to the invention.

This device is particularly suitable for propelling this type of composition, and likewise, the composition is particularly suited to this device. The assembly thus forms an advantageous combination.

It has been known for a long time that the ultra-violet (UV) rays emitted by the sun have not only visible short-term effects (sunburn, tanning), but also cause long-term damage, in particular skin cancer. Special sunscreens particularly intended for children have been developed, since it has been shown that childhood is the key period in terms of memorization of the damage caused by incomplete photoprotection. Historically, the first photoprotective agents to be used were oil-soluble or water-soluble synthetic screening agents. These synthetic (organic) screening agents have the property of absorbing the UV rays instead of the skin, but have a certain number of drawbacks, in particular a protection factor that reduces over time and according to the sunshine (photo-instability), penetration into the skin, which raises the question of their fate in the body (especially in the case of children, who are more sensitive to toxic effects) and finally an absorption of UVB or UVA but, all too rarely, of both simultaneously. In recent years, mineral (or inorganic) screening agents have been used, especially for antisun products for children. These agents are generally titanium dioxide and zinc oxide, which have the appearance of a white powder consisting of small particles of these pigments. Although these already-known products have certain advantages, in particular absence of penetration into the skin, stability in sunlight and over time, and also total inertness with respect to skin reactions (irritations, allergies, etc.), there remained hitherto a major drawback for the user, namely a very white covering appearance and difficulty in spreading, preventing effective protection from being obtained since either the user could not manage to apply the product correctly to all the areas to be protected, or he did not even attempt to obtain such an application, given the unesthetic appearance and the impractical nature of these very white and pasty covering products, resulting in incomplete photoprotection of the areas to be protected in priority, i.e. the face, the neck, the shoulders, the hands and the feet.

In point of fact, the substantial viscosity of the existing products results from a problem of nonuniform dispersion of the mineral pigments in the known excipients. Specifically, since nonuniform dispersion not only prevents good photoprotection (high protection factor) and also broad-spectrum photoprotection (UVB, short UVA and long UVA) from being obtained, even in areas where the application of the product may appear correct to the user, it was necessary hitherto to compensate for this lack of homogeneity by increasing the amount of mineral pigment and thus the viscosity of the product.

A milk sunscreen containing an inorganic screening agent and the same type of silicone emulsifier with a glucose component is known from document WO 01/74294 in the name of the Applicant. However, the formulations described therein do not allow application via a spray pump or via a jet pump. Specifically, the viscosity reached by the formulations described therein is about 10 times higher than that which the compositions of the present invention can obtain. Comparative examples are collated hereinbelow, to allow a clear appreciation of the improvements afforded over the compositions of the prior art.

It has now been found, entirely surprisingly and unexpectedly, that the combination of a mineral screening agent and of the organic screening agent having the international nonproprietary name (INN) methylenebis(benzotriazolyl)tetramethylbutylphenol with certain emulsifiers makes it possible to obtain a cosmetic or dermatological composition that simultaneously has very good fluidity and excellent photoprotection, which furthermore is broad spectrum, while at the same time maintaining good stability.

The cosmetic or dermatological composition according to the invention thus gives, firstly, outstanding surface protection, reflected by protection factors that may be greater than 30, as illustrated by the examples below, while at the same time showing very good fluidity, i.e. a viscosity that may be less than 10 Pa·s (10 000 centipoises) at 25° C.

In addition, not only does this cosmetic or dermatological composition no longer have the drawback of a viscous or even pasty appearance, but also it is totally transparent (no whitening effect), which is in cosmetic terms a crucial advantage for a high-protection antisun product. What is more, it has the major advantage of being able to be adapted for application by a device of propulsion (jet or spray) pump bottle type, more particularly by virtue of the particular pump described below operating by manual propulsion.

One subject of the present invention is thus a cosmetic or dermatological composition for protecting against ultraviolet rays, based on a mixture of mineral screening agents and the organic screening agent having the INN name methylenebis(benzotriazolyl)tetra-methylbutylphenol, characterized in that it is in the form of a water-in-oil emulsion and in that it contains at least one emulsifier chosen from the group consisting of silicone derivatives with a glucose component comprising between 2 and 10 glucose units, the particulate inorganic screening agent being uniformly dispersed in the water-in-oil emulsion and its mean particle size being between 1 and 100 nano-meters, and the particulate inorganic screening agent being present in a proportion of from 4% to 40% by weight.

An assembly for applying a cosmetic or dermatological composition for protecting against ultraviolet rays is also proposed according to the invention, comprising such a composition and a container for this composition, said container consisting of a reservoir and a manually-driven propulsion pump, characterized in that the composition is in the form of a water-in-oil emulsion and in that it contains at least one emulsifier chosen from the group consisting of silicone derivatives with a glucose component comprising between 2 and 10 glucose units, the particulate inorganic screening agent being uniformly dispersed in the water-in-oil emulsion and its mean particle size being between 1 and 100 nano-meters, and the particulate inorganic screening agent being present in a proportion of from 4% to 40% by weight.

A cosmetic skin-treatment process for protecting the skin against the harmfulness of and attack by ultraviolet rays is also proposed according to the invention, which consists in diffusing from a propulsion pump bottle an effective amount of a cosmetic composition, using such an assembly.

The use of such compositions for the manufacture of compositions intended to be diffused via a propulsion pump bottle onto the skin for the purpose of protecting the skin against the harmfulness of and attack by ultraviolet rays is also proposed according to the invention.

The use of a device of propulsion pump bottle type for applying such a composition to the skin is also proposed according to the invention.

The organic screening agent having the INN name methylenebis(benzotriazolyl)tetramethylbutylphenol is also known under the brand name Tinosorb M.

For the purposes of the present invention, the term “silicone derivatives with a glucose component” means any silicone derivative comprising between 2 and 10 glucose units. (C₂-C₃₀)Alkylsilicones and amino(C₂-C₃₀)alkylsilicones are preferred as silicone derivative. Thus, among the silicone derivatives with a glucose component according to the present invention, mention may be made especially of the derivatives obtained by reacting dimethicone polymers with glucose polymers. Examples of dimethicone polymers that may be mentioned include amino bispropyl dimethicone, amino-propyl dimethicone, amodimethicone, cetyl dimethicone, hexyl dimethicone, octyl dimethicone and stearyl dimethicone.

In one particular embodiment of the composition according to the invention, the silicone derivative with a glucose component is the product of reacting octyl dimethicone with a glucose polymer, known as ethylhexyl dimethicone ethoxy glucoside (INCI name ethylhexyl dimethicone ethoxy glucoside No. 528 in the International Cosmetic Ingredients Dictionary and Handbook, 8th edition). Another registered name is silicone polyglucoside.

According to the present invention, the composition may also comprise at least one other standard emulsifier, especially cyclodimethicone.

In one particular embodiment of the composition according to the invention, the proportion of emulsifier is between about 2% and about 30% by weight relative to the total weight of the composition.

In the context of the present invention, the term “mineral screening agent” means a particulate inorganic screening agent chosen from the group consisting of titanium dioxide and zinc oxide, and mixtures thereof. The mineral screening agents may also be coated with ingredients of diverse nature (fatty acids, silicones, metals, etc.).

Among the titanium dioxides used as inorganic screening agent, mention may be made of hydrophilic or hydrophobic titanium dioxides, preferably iron-doped titanium dioxides. Among the commercially available titanium dioxides, a hydrophilic dioxide that may be mentioned is Titanium dioxide P 25 S (75% anatase and 25% rutile), a hydrophobic dioxide that may be mentioned is Titanium dioxide T 805, an iron-doped hydrophilic dioxide that may be mentioned is Titanium dioxide PF 2, and an iron-doped hydrophobic dioxide that may be mentioned is Titanium dioxide T 817, from the company Degussa. The titanium oxides used in the present invention may also be synthesized according to the Aerosil® process developed by the company Degussa. This process especially involves the co-calcination of the titanium and iron chlorides TiCl₄ and FeCl₃, respectively, at high temperature and in the presence of an oxohydrogen flame. The synthesized iron-doped titanium oxides have a mean BET surface area of 50 m²/g and a mean particle size of 21 nm.

Also, it has been demonstrated that the UVA- and UVB-absorbing capacity of the titanium oxides increases as their dispersion increases. The introduction of iron during the synthetic process leads to the formation of mixed iron-titanium oxides, in which the titanium oxide is more dispersed. Finally, the insertion of iron into a titanium oxide phase, mainly of anatase type, leads to a change in the semiconductive properties of the titanium oxide and thus of its photochemical properties.

In one particular embodiment of the composition according to the invention, the particulate inorganic screening agent is a mixture of coated titanium dioxide and zinc oxide.

According to the present invention, the fatty phase may also comprise other cosmetically or dermatologically acceptable fatty substances, especially animal, plant or mineral oils and analogs thereof, fatty alkyl benzoates and fatty acid triglycerides.

According to the invention, the aqueous phase comprises water and cosmetically or dermatologically acceptable hydrophilic compounds, among which mention may be made of glycerol, and optionally organic solvents, for instance water-soluble lower alcohols.

The cosmetic or dermatological composition according to the invention has a viscosity of less than 10 Pa·s (10 000 centipoises) at 25° C., measured with a Brookfield viscometer.

As regards the impairment of the DNA of the skin cells by UV rays, its essential repair, which will limit the perpetuation of the damage, is controlled by the protein P53 which orders cell repair or destruction (apoptosis). During exposure to sunlight, the protein P53 may become ineffective by mutation in the gene, resulting in the proliferation of abnormal cells, the recognition and control of which are governed by the immune system (Langerhans cells), which is the final defense before the tumor process.

As regards photoimmunosuppression, recent studies have shown that UV rays impair the essential cells of skin immunity, the Langerhans cells. They destroy them or create dysfunctions by inhibiting their action of recognition and protection against foreign bodies (bacteria, viruses, tumors or allergens).

These two attacks deep down in the skin, caused by the action of UV rays, may result in the formation of skin cancers.

Now, as it turns out, although most antisun products protect more or less well against UV rays, by preventing sunburn, the most recent studies suggest that their efficacy in particular against the photoimmunosuppression induced by UV rays is only partial and thus a chronic immunosuppression may develop in the absence of visible sunburn and despite the application of these antisun products.

Epidemiological studies have even shown that the risk of skin cancer is higher in the case of sunscreen users, this paradox possibly being explained by the fact that the absence of sunburn would allow longer exposures and thus promote the invisible damage of UV radiation.

Thus, the composition according to the invention may also comprise at least one agent for protecting against the immunosuppression induced by ultraviolet rays, chosen from the group consisting of Aloe vera (extract of Aloe barbadensis), vitamin E and the unsaponifiable matter of soybean oil, and mixtures thereof, in a proportion advantageously of between about 0.05% and about 5% by weight relative to the total weight of the composition.

The cosmetic or dermatological composition according to the invention may also comprise at least one agent for protecting the DNA of skin cells, chosen from the group consisting of isoflavones and/or zinc salts, in a proportion advantageously of between about 0.01% and about 1% by weight relative to the total weight of the composition, said salt advantageously being zinc gluconate.

The “isoflavones” that may be used according to this particular embodiment of the present invention are obtained via chemical synthesis or are natural substances extracted from natural products, especially from plants such as soybean, clover, lupin, apple pips, etc. The topical compositions according to the present invention very often contain, as isoflavones, a mixture of different isoflavones, but they may also be present in pure form in the context of the present invention. Moreover, the aglycone forms of the isoflavones and the glycosylated forms thereof are distinguished. These various forms are usually present as a mixture. They are illustrated by the following formulae.

Aglycone forms, of formula: 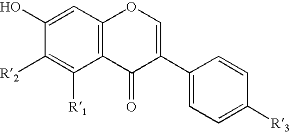
in which R′₁ represents a hydrogen atom or a hydroxyl group, R′₂ represents a hydrogen atom or a methoxy group and R′₃ represents a hydroxyl group.

Advantageously, according to the present invention, R′₁, R′₂ and R′₃ represent:

| R′₁ | R′₂ | R′₃ | Compound name |
|---|---|---|---|
| H | H | OH | Daidzeine |
| OH | H | OH | Genisteine |
| H | OCH₃ | OH | Glyciteine |

Glycosylated forms, of formula: 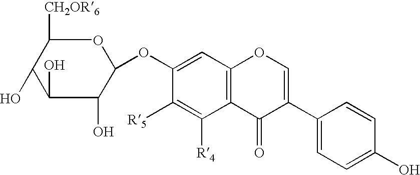
in which R′₄ represents a hydrogen atom or a hydroxyl group, R′₅ represents a hydrogen atom or a methoxy group and R′₆ represents a hydrogen atom.

Advantageously, according to the present invention,

| R′₄ | R′₅ | R′₆ | Compound name |
|---|---|---|---|
| H | H | H | Daidzine |
| OH | H | H | Genistine |
| H | OCH₃ | H | Glycitine |

The glycosylated forms of the isoflavones are the ones most abundant in nature.

Natural isoflavones such as genisteine (1), daidzeine or glyciteine are preferred as isoflavones.

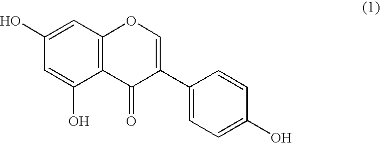

In particular, genisteine or 4,5,7-trihydroxy-isoflavone, which may be used according to the present invention, may be a product of plant origin and especially from soybean, with an 85% to 90% by weight titer of genisteine, especially the product sold by the company Buckton Scott under the name “85%-titer genisteine”.

Thus, the cosmetic or dermatological composition according to the invention also has, in addition to this broad-spectrum photoprotection obtained via the specific mineral screening agent-emulsifier combination, the advantage of giving deep-down protection against the impairment of the DNA of the skin cells and more particularly against the phenomenon of photoimmunosuppression.

Needless to say, the composition according to the invention may also contain one or more standard lipophilic or hydrophilic cosmetic adjuvants, especially those already usually used in the manufacture and production of cosmetic or dermatological antisun compositions.

Thus, the cosmetic or dermatological composition according to the invention may also comprise at least one adjuvant chosen from the group consisting of ionic or nonionic thickeners, softeners, antioxidants, opacifiers, stabilizers, emollients, insect repellents, moisturizers, vitamins, fragrances, preserving agents, fillers, sequestrants and dyes, and mixtures of these compounds.

The cosmetic or dermatological composition according to the invention may be prepared via any method known to those skilled in the art, especially by mixing together the various ingredients.

The cosmetic or dermatological composition according to the invention may be in the form of a cream, a milk, a gel, a cream-gel or any other form generally suited to packaging in a spray pump or a jet pump and intended to be used in cosmetics or dermatology for the topical application of a water-in-oil emulsion, especially those that are usually suitable for cosmetic or dermatological antisun compositions.

Another subject of the present invention lies in a cosmetic skin-treatment process for protecting the skin against the harmfulness of and attack by ultraviolet rays and which consists in diffusing onto the skin via a spray pump or jet pump an effective amount of a cosmetic composition as defined above.

The compositions according to the invention may thus be used in the treatment of pathologies associated with UVA and UVB, especially erythema, acne, aging, immunosuppression, inflammation and also the aggravation of other dermatological pathologies (acne, rosacea, etc.).

The device of propulsion pump type that is especially recommended herein, and which proves to be most specifically advantageous for the present composition, is as follows.

This pump is illustrated more specifically in the single FIGURE appended to the present description. The present pump is a jet pump, i.e. it delivers a flow of said antisun composition in a relatively targeted form.

However, use of the pump by moving during this propulsion allows a certain amount of spreading of the product onto the skin and especially the desired distribution.

This pump is designed around the use of an inter-mediate chamber 10 of substantially cylindrical form and coaxial with the bottle assembly (reservoir not shown).

This intermediate chamber is delimited at its upper and lower ends by respective nonreturn devices. The bottom nonreturn device, i.e. the one adjacent to the reservoir, is composed of a bead 20 lying on a funnel neck when returned into this position by an associated spring.

The top nonreturn device also operates on the principle of an obstructing member that can move vertically in the chamber. The obstructing member is herein a piston 30, in the form of a circular seal, surrounding a nozzle 40, which is herein in the form of a movable needle intended to occupy the volume of the chamber when pressed down by the user, the nozzle then being able to convey the composition in the direction of the outlet.

The principle of this nonreturn member 30 is based on the fact that the end apertures of the nozzle 40, which has slid vertically during the actuation of the pump, are then freed by this circular piston 30 in order to allow the composition, lying previously in the chamber 10, to enter the nozzle 40. The piston 30 is thus slightly offset relative to these apertures.

In contrast, when the nozzle 40 is released by the user, i.e. returned upward under the pressure of a helical spring, the piston 30 has a tendency by friction to regain its position before the inner circulation apertures of the nozzle 40, thus making the nozzle leaktight and, under the upward movement of this nozzle, the piston 30 then causes suction of the composition from the reservoir into the intermediate chamber 10.

Also, when the nozzle 40 regains its rest position by extension for the top part of the pump, the piston 30 also regains the obstruction position, preventing any leakage of composition out of the nozzle 40 and out of the pump.

In other words, to summarize the functioning of this pump, the top nonreturn member 30 frees circulation apertures of a mechanical member that occupies the inner space of the intermediate chamber 10 filled with composition, allowing the composition to rise inside this member 40. In contrast, this nonreturn member 30 prevents the composition from leaving the nozzle and heading toward the intermediate chamber when the nozzle moves upward, thus taking the composition with it.

The top nonreturn member is thus a member associated with a movable component for dispensing the composition.

The present pump is mounted on a screw means intended for a relatively standard reservoir.

However, for the propulsion of the composition described in detail hereinabove and hereinbelow, this pump has been rated to give a particularly effective jet, and to prevent subsequent dysfunction of such a pump, for example by clogging or backflow, said phenomena corresponding to poor circulation therein.

For this propulsion efficacy, the pump has particular sizes in different areas of circulation of the composition.

Thus, by following the flow of the composition from the reservoir to the outlet nozzle, the following elements have been adopted, and have been found to be particularly suited to the present composition and more generally to this type of fluidity in the context of antisun compositions.

Thus, a dip tube 60 extending into the reservoir has an inside diameter increased herein to 3.70 mm, which is particularly suitable for creams of less than 10 Pa·s (10 000 centipoises) at 25° C.

The bead 20 has a diameter of 3 mm, and the pump body has an outside diameter of 8.6 mm, the corresponding intermediate chamber 10 thus having a smaller diameter substantially equal to 8 mm.

The latter arrangements are more particularly favorable for eliminating risks of clogging of cream at these points in the course of successive dispensing.

The nozzle 40 is returned upward by a helical spring, described previously, the compression force of which is between 35 and 40 newtons, for a sufficient suction power. The piston 30 is, itself also, returned to its obstruction position by a helical spring with a force of between 35 and 40 newtons.

It will be recalled that the total return force is preferentially greater than 35 newtons, for sufficient suction of the composition present.

More specifically, the bottom spring is rated herein at 40 newtons and the spring associated with the piston 30, and also returning the nozzle 40 upward via an associated bearing neck, itself has a force of 35 newtons.

In total, this pump has an intermediate chamber 10 volume very advantageously of between 100 and 200 μl and even more preferentially between 150 and 200 μl.

Finally, the nozzle 50 has a diameter of between 4 and 5 mm, allowing emptying of the intermediate chamber 10 without causing any backflow, i.e. without giving rise to spurious flows in the direction opposite to the outlet direction.

Besides maintaining good functioning of the pump during its service life, this nozzle 50 is found to be able to deliver a dose of cream that is necessary and sufficient for good antisun protection and for sufficient application of the present composition to the skin.

The precision of the condensed unidirectional jet obtained allows the delivery of a large amount of this composition to a desired area, promoting optimum antisun protection.

Although the present pump is directed toward the delivery of a jet, it is also adapted, on condition of making a few readily accessible adjustments by virtue of the usual knowledge of a person skilled in the art, to the delivery of a spray. A spray, or spraying, is directed toward application over a wider area.

The examples that follow illustrate the invention without, however, limiting it to these particular embodiments.

Unless otherwise indicated, the percentages indicated in the examples that follow are percentages of total weight of the composition.

### EXAMPLE 1

### Procedure for Antisun Products

1—Preparation of the Fatty Phase

The fatty phase (mineral pigments, emulsifier and oil) is placed in a reactor and homogenized with recycling to obtain good dispersion.

This phase is heated to 60° C.

2—Preparation of the Aqueous Phase

The gelling agent is dispersed in the aqueous phase containing the electrolytes, in a flask with stirring, and the active agents are then added.

This phase is heated to 60° C. Methylenebis-(benzotriazolyl)tetramethylbutylphenol is then introduced.

3—Emulsification

The aqueous phase is poured slowly (30 minutes) into the oily phase at 60° C., while stirring fairly vigorously (using a turbomixer or a doctor blade) and the appearance of the emulsion is inspected.

The mixture is homogenized for 30 minutes with recycling while cooling the product to 25° C.

The product is emptied out and then checked after standing for 1 hour to obtain an indication of its viscosity. Perfect uniformity of the emulsion is checked.

4—Measurement of the Viscosity

The viscosity is measured at 25° C. using a Brookfield viscometer.

| | | Viscosity at 25° C. |
|---|---|---|
| Emulsifier | PF | Pa · s (cp) |
| PA (cetyl dimethicone polyols | 35 | 200 (200 000) |
| PA (cetyl dimethicone polyols) | 20 | 60 (60 000) |
| PA (example 1) | 25 | 15 (15 000) |
| Invention (example 2) | 32 | 7 (7000) |
| Invention (example 3) | 40 | 10 (10 000) |

Thus, for a similar protection factor (PF), the compositions according to the invention have a markedly lower viscosity than that of the compositions of the prior art (PA).

### EXAMPLE 1

### Formulation with a Protection Factor of Greater than 25 (Comparative Example)

| Ingredients | weight % | |
|---|---|---|
| Water | 30 to | 60 |
| Pentaerythrityl tetraoctanoate | 15 to | 30 |
| Titanium dioxide | 1 to | 10 |
| Cyclomethicone | 1 to | 10 |
| Zinc oxide | 1 to | 10 |
| (C12-C15)alkyl benzoate | 1 to | 10 |
| 2-Heptadecadienylfuran | 0.1 to | 10 |
| 4,5,7-Trihydroxyisoflavone | 0.01 to | 10 |
| Glycerol | 1 to | 10 |
| Dicaprylyl ether | 1 to | 10 |
| Cyclopentasiloxane | 1 to | 10 |
| Ethylhexyl dimethicone ethoxy glucoside | 1 to | 10 |
| Propylene glycol dioctanoate | 1 to | 10 |
| Sodium chloride | 1 to | 5 |
| PEG-45/dodecyl glycol copolymer | 1 to | 5 |
| PEG-30 dipolyhydroxystearate | 1 to | 5 |
| Unsaponifiable matter of soybean oil | 1 to | 5 |
| Dextrin palmitate | 1 to | 5 |

| | |
|---|---|
| Phenoxyethanol | 0.5 |
| Extract of Aloe barbadensis | 0.2 |
| Methylparaben | 0.16 |
| Zinc gluconate | 0.08 |
| Butylparaben | 0.06 |
| Ethylparaben | 0.04 |
| Propylparaben | 0.02 |

### EXAMPLE 2

### Formulation with a Protection Factor of Greater than 30

| Ingredients | weight % | |
|---|---|---|
| Water | 20 to | 50 |
| Pentaerythrityl tetraoctanoate | 15 to | 30 |
| Titanium dioxide | 1 to | 10 |
| Cyclomethicone | 1 to | 10 |
| Zinc oxide | 1 to | 10 |
| Methylenebis(benzotriazolyl)tetramethyl- | 1 to | 5 |
| butylphenol | | |
| (C12-C15)alkyl benzoate | 1 to | 10 |
| 2-Heptadecadienylfuran | 0.1 to | 10 |
| 4,5,7-Trihydroxyisoflavone | 0.01 to | 10 |
| Glycerol | 1 to | 10 |
| Dicaprylyl ether | 1 to | 10 |
| Cyclopentasiloxane | 1 to | 10 |
| Ethylhexyl dimethicone ethoxy glucoside | 1 to | 10 |
| Propylene glycol dioctanoate | 1 to | 10 |
| Sodium chloride | 1 to | 5 |
| PEG-45/dodecyl glycol copolymer | 1 to | 5 |
| PEG-30 dipolyhydroxystearate | 1 to | 5 |
| Unsaponifiable matter of soybean oil | 1 to | 5 |
| Dextrin palmitate | 1 to | 5 |

| | |
|---|---|
| Phenoxyethanol | 0.5 |
| Extract of Aloe barbadensis | 0.2 |
| Methylparaben | 0.16 |
| Zinc gluconate | 0.08 |
| Butylparaben | 0.06 |
| Ethylparaben | 0.04 |
| Propylparaben | 0.02 |

### EXAMPLE 3

### Formulation with a Protection Factor of Greater than 40

| Ingredients | weight % | |
|---|---|---|
| Water | 20 to | 50 |
| Pentaerythrityl tetraoctanoate | 15 to | 30 |
| Titanium dioxide | 1 to | 10 |
| Cyclomethicone | 1 to | 10 |
| Zinc oxide | 1 to | 10 |
| Methylenebis(benzotriazolyl)tetramethyl- | 1 to | 10 |
| butylphenol | | |
| (C12-C15)alkyl benzoate | 1 to | 10 |
| 2-Heptadecadienylfuran | 0.1 to | 10 |
| 4,5,7-Trihydroxyisoflavone | 0.01 to | 10 |
| Glycerol | 1 to | 10 |
| Dicaprylyl ether | 1 to | 10 |
| Cyclopentasiloxane | 1 to | 10 |
| Ethylhexyl dimethicone ethoxy glucoside | 1 to | 10 |
| Propylene glycol dioctanoate | 1 to | 10 |
| Sodium chloride | 1 to | 5 |
| PEG-45/dodecyl glycol copolymer | 1 to | 5 |
| PEG-30 dipolyhydroxystearate | 1 to | 5 |
| Unsaponifiable matter of soybean oil | 1 to | 5 |
| Dextrin palmitate | 1 to | 5 |

| | |
|---|---|
| Phenoxyethanol | 0.5 |
| Extract of Aloe barbadensis | 0.2 |
| Methylparaben | 0.16 |
| Zinc gluconate | 0.08 |
| Butylparaben | 0.06 |
| Ethylparaben | 0.04 |
| Propylparaben | 0.02 |

### EXAMPLE 4

### Method for Determining the Sun Protection Factor

The level of sun protection involves the erythemal response of the skin to ultraviolet radiation. It is expressed as the sun protection factor (SPF), which is the ratio of the energies required to induce a minimum erythemal response on the skin of volunteers who are or are not protected with the test product, using ultra-violet radiation generally provided by an artificial source.

The method used is that of the COLIPA (European Cosmetic Toiletry and Perfumes Association) described in “Method for determining the sun protection factor”, Ref.: 94/289, October 1994).

The lowest dose that produces an erythema, known as the minimum erythemal dose (MED) is determined for each volunteer, either without protection (MEDn), or with protection (MEDp), and the SPF is calculated as being the ratio MEDp/MEDn.

The compositions according to the invention have protection factors that may be up to 40.

1. A cosmetic or dermatological composition for protecting against ultraviolet rays, based on a mixture of mineral screening agents and the organic screening agent having the INN name methylenebis(benzotriazolyl)tetramethylbutylphenol, wherein said composition is in the form of a water-in-oil emulsion and said composition contains at least one emulsifier chosen from the group consisting of silicone derivatives with a glucose component comprising between 2 and 10 glucose units, the particulate inorganic screening agent being uniformly dispersed in the water-in-oil emulsion and its mean particle size being between 1 and 100 nanometers, and the particulate inorganic screening agent being present in a proportion of from 4% to 40% by weight.

2. The cosmetic or dermatological composition as claimed in claim 1, wherein the silicone derivative is chosen from (C₂-C₃₀)alkylsilicones and amino(C₂-C₃₀)alkylsilicones.

3. The cosmetic or dermatological composition as claimed in claim 2, wherein the silicone derivative with a glucose component is ethylhexyl dimethicone ethoxy glucoside or silicone polyglucoside.

4. The cosmetic or dermatological composition as claimed in claim 1, wherein the cosmetic composition also comprises cyclodimethicone.

5. The cosmetic or dermatological composition as claimed in claim 1, wherein the proportion of emulsifier is between about 2% and about 30% by weight relative to the total weight of the composition.

6. The cosmetic or dermatological composition as claimed in claim 1, wherein the mineral screening agent is a particulate inorganic screening agent chosen from the group consisting of titanium dioxide and zinc oxide, and mixtures thereof.

7. The cosmetic or dermatological composition as claimed in claim 1, wherein the particulate inorganic screening agent is hydrophobic titanium dioxide.

8. The cosmetic or dermatological composition as claimed in claim 1, wherein it has a viscosity of less than 10 Pa·s (10 000 centipoises) at 25° C.

9. The cosmetic or dermatological composition as claimed in claim 1, wherein it also comprises at least one agent for protecting against the immunosuppression induced by ultraviolet rays, chosen from the group consisting of Aloe vera, vitamin E and the unsaponifiable matter of soybean oil, and mixtures thereof.

10. The cosmetic or dermatological composition as claimed in claim 9, wherein the proportion of agent for protecting against the immunosuppression induced by ultraviolet rays is between about 0.05% and about 5% by weight relative to the total weight of the composition.

11. The cosmetic or dermatological composition as claimed in claim 1, wherein it also comprises at least one agent for protecting the DNA of skin cells, chosen from the group consisting of isoflavones and/or zinc salts.

12. The cosmetic or dermatological composition as claimed in claim 11, wherein the agent for protecting skin cells is zinc gluconate.

13. The cosmetic or dermatological composition as claimed in claim 11, wherein the proportion of agent for protecting skin cells is between about 0.01% and about 1% by weight relative to the total weight of the composition.

14. The cosmetic or dermatological composition as claimed in claim 1, wherein it also comprises at least one adjuvant chosen from the group consisting of ionic or nonionic thickeners, softeners, antioxidants, opacifiers, stabilizers, emollients, insect repellents, moisturizers, vitamins, fragrances, preserving agents, fillers, sequestrants and dyes, and mixtures thereof.

15. An assembly for applying a cosmetic or dermatological composition for protecting against ultraviolet rays, comprising such a composition and a container for this composition, said container consisting of a reservoir and a manually-driven propulsion pump, wherein the composition is in the form of a water-in-oil emulsion and in that it contains at least one emulsifier chosen from the group consisting of silicone derivatives with a glucose component comprising between 2 and 10 glucose units, the particulate inorganic screening agent being uniformly dispersed in the water-in-oil emulsion and its mean particle size being between 1 and 100 nanometers, and the particulate inorganic screening agent being present in a proportion of from 4% to 40% by weight.

16. The assembly as claimed in claim 15, wherein the pump is a pump with an intermediate chamber and a sliding member capable of occupying the inner volume of the intermediate chamber to deplete the composition present in this intermediate chamber.

17. The assembly as claimed in claim 15, wherein the pump is a pump with an intermediate chamber surrounded by two nonreturn devices oriented in the direction of outlet of the reservoir, and wherein one of the nonreturn devices, located adjacent to the reservoir, is an obstruction bead.

18. The assembly as claimed in claim 15, wherein the pump is equipped with an outlet nozzle the composition circulation space of which has a diameter of between 4 and 5 mm.

19. The assembly as claimed in claim 15, wherein the pump has an intermediate chamber with a volume of between 100 and 200 μl.

20. The assembly as claimed in claim 15, wherein the pump has one or more return members for filling the intermediate volume, producing in total a force of greater than 35 N.

21. The assembly as claimed in claim 15, wherein the pump has an intermediate chamber of cylindrical type, the diameter of which is about 8 mm.

22. The assembly as claimed in claim 15, wherein the pump is equipped with a dip tube with an inside diameter of about 3.7 mm.

23. (canceled)

24. A method for protecting the skin against the harmfulness of and attack by ultraviolet rays consisting of diffusing from a propulsion pump bottle an effective amount of the composition, using an assembly in accordance with claim 15, by applying the composition to the skin of a patient in need thereof.

25. (canceled)

26. The cosmetic or dermatologic composition as claimed in claim 7, wherein the hydrophobic titanium dioxide is coated.
